(19) United States
(12) Patent Application Publication
(10) Pub. No.: US 2007/0032443 A1
(43) Pub. Date: Feb. 8, 2007

(54) Therapy for Alzheimer's disease

(76) Inventors: Jaeseob Kim, Daejeon (KR); Eunkyung Bae, Daejeon (KR); Jungmo Kim, Seoul (KR)

Correspondence Address: MEDLEN & CARROLL, LLP, Suite 350, 101 Howard Street, San Francisco, CA, 94105, US

(21) Appl. No.: 11/364,739

(22) Filed: Feb. 28, 2006

Related U.S. Application Data
(63) Continuation of application No. 11/195,032, filed on Aug. 2, 2005, now abandoned.

(51) Int. Cl. A61K 48/00; A61K 38/16; A61K 31/70; A61K 31/675; A61K 31/663

(52) U.S. Cl. 514/44; 514/89; 514/102; 514/8; 514/25; 514/560

(57) ABSTRACT

The present invention relates to newly identified methods and compositions for modulating the cellular processing of beta-amyloid precursor protein (“APP”) and for the prevention or treatment of diseases associated with abnormal APP processing, such as Alzheimer's disease (“AD”). The invention also relates the identification of molecular pathways heretofore unknown to be involved in APP processing. The invention also relates to the prevention or treatment of AD disease symptoms using medicaments that modify the activity of novel molecular targets involved in APP processing.

### FIELD OF THE INVENTION

The present invention relates to newly identified methods and compositions for modulating the cellular processing of beta-amyloid precursor protein (“APP”) and for the prevention or treatment of diseases associated with abnormal APP processing, such as Alzheimer's disease (“AD”). The invention also relates the identification of molecular pathways heretofore unknown to be involved in APP processing that provide novel molecular targets for pharmacological intervention. The invention also relates to the prevention or treatment of AD disease symptoms using medicaments that modify the activity of novel molecular targets involved in APP processing.

### BACKGROUND OF THE INVENTION

AD is the most common cause of age-related dementia and is a major cause of disability and death in the elderly (Hock and Lamb, Trends Genet. 17:S7 (2001); Tanzi and Bertram, Neuron 32:181 (2001)). This disease, for which there is currently no effective cure, is a long-progressing, neurodegenerative disorder of the central nervous system characterized by increasingly debilitating, global cognitive defects including loss of memory, language deficits, and impaired judgment and abstract reasoning.

Post-mortem examination of brain tissue from AD patients reveals proteinaceous filaments comprising extracellular amyloid senile plaques and intraneuronal neurofibrillary tangles (Selkoe, Proc. Natl. Acad. Sci. USA 98:11039 (2001)). These filaments comprise aggregates of a variant of the beta-amyloid precursor protein (“APP”) called amyloid beta-protein (“A-beta”). Mutations or polymorphisms in the APP gene are found in patients with familial AD (“FAD”) and are sufficient to increase in A-beta production in cultured cells, thus establishing a direct link between human gene variants and the disease pathology (Chapman et al., Trends Genet. 17:254 (2001)).

The inheritance of predisposing genetic factors appears to play a major role in the etiology of AD. Three other contributing genes have also been implicated: presenilin-1, presenilin-2 (PS-1 and PS-2, respectively, and together referred to as “PS”), and apolipoprotein E (“APOE”) (Tanzi and Bertram, 2001). Furthermore, various biochemical and animal model-based experiments have shown that the expression of different variants of each of these genes can significantly contribute to A-beta accumulation. However, these genes account for less than thirty percent of the genetic variance in age of onset for AD, and recent estimates suggest that numerous additional AD genes may exist (Tanzi and Bertram, 2001).

The A-beta protein is a fragment resulting from a sequence of proteolytic cleavage steps of the integral membrane APP protein. The alpha-secretase protein cleaves APP and releases a soluble, eighty-three amino acid-long C-terminal fragment (C83). This fragment does not contribute to A-beta accumulation. Alternatively, the activity of the beta-secretase (“BACE”) protein cleaves APP into a membrane-bound, ninety-nine amino acid-long C-terminal fragment (C99). C99 is further proteolytically processed in a heterogeneous manner by the gamma-secretase protein into two different fragments: A-beta-40, which does not readily form plaques, and A-beta-42, which is prone to form fibrils and is the major A-beta protein variant found in cerebral plaques (Esler and Wolfe, Science 293:1449 (2001)).

Gamma-secretase, the critical component of the A-beta-42 production machinery, is a multiprotein complex with the pharmacologic characteristics an aspartyl protease (Esler and Wolfe, 2001). Its active site is thought to comprise the protein products of the PS genes. The PS proteins co-immunoprecipitate with gamma-secretase and heighten its ability to produce A-beta-42. Furthermore, cells from PS-1-deficient mouse embryos exhibit a marked reduction in gamma-secretase activity (Esler and Wolfe, 2001). Additional evidence also supports the model that the PS proteins form the active site of gamma-secretase (Selkoe, 2001). Mutant alleles of both PS genes are the most common known contributors to FAD (Kopan and Goate, Neuron 33:321 (2002)).

Gamma-secretase is also a critical component of an evolutionarily conserved signaling mechanism mediated by the Notch family of transmembrane receptors that is critical to animal development (Hardy and Israel, Nature 398:466 (1999); Kopan and Goate, 2002). In many animals, Notch signaling facilitates the specification of proper fates among equivalent cells. Ligand binding triggers the proteolytic cleavage of an extracellular Notch domain, creating a substrate for proteolysis by gamma-secretase that releases the Notch intracellular domain. This intracellular domain translocates to the nucleus, where it interacts with a transcriptional activating protein, and together they affect the transcription of a number of target genes. Therefore, the PS genes and their encoded proteins play critical roles in both AD pathogenesis and in animal development.

Compounds that inhibit the activity of PS activity have the potential to ameliorate the formation of cerebral plaques in AD by preventing the production of neurotoxic A-beta-42. Although the PS genes are important genetic factors in the progression of AD, they account for only a fraction of all AD in patients; FAD accounts for only approximately 4-8% of all AD cases (Chapman et al., 2001). Other genes must therefore contribute to at least 92% of all other forms of human AD. It is likely that other genes that contribute to predisposition to AD progression in humans will encode proteins that influence PS activity and the production of A-beta-42. Therefore, it is important to identify additional molecular components of A-beta-42-specific production machinery in order to discover methods of inhibiting the accumulation of A-beta-42, most preferably without disrupting Notch signaling.

Thus, the art is in need of additional genes and proteins that are involved in the cellular processing of APP because of their action in the pathophysiology of AD, and to better identify subjects with AD, subjects likely to develop AD, compounds that regulate AD, and targets for therapeutic regulation of AD.

### SUMMARY OF THE INVENTION

The present invention provides a method for the identification and use of compounds involved in the modulation of the cellular processing of APP, regulation of biological pathways associated with APP processing, or regulation of gene expression or protein function of a gene or protein associated with APP processing. Moreover, the present invention provides a method for prevention or treatment of diseases associated with the accumulation of APP cleavage products, such as AD, by using medicaments that modulate APP processing. The present invention may involve modulation of AP processing within cells that exist in vitro (e.g., in tissue culture, on a cell array, etc.), ex vivo (e.g., in an isolated tissue), or in vivo (e.g., in an organism). The medicament of the present invention is not limited to a specific composition but rather encompasses any form of pharmaceutical agent that may be suitable for administration to a subject. Agents include antibodies, antisense molecules (e.g., antisense oligonucleotides, siRNAs, etc.), small molecule drugs, peptides, and the like. In some preferred embodiments, the agent is part of a compound library. The present invention is not limited by the method in which modulation of APP processing, regulation of biological pathways associated with APP processing, or regulation of gene expression or protein function of a gene or protein associated with APP processing associated with AD are identified. Identification can be direct (e.g., conducing a memory test, measuring plaque formation, measuring an enzymatic activity, measuring the amount of an expression product in a cell, etc.) or indirect.

In some embodiments, agents identified that result in the modulation of a symptom of AD, regulation of a biological pathway associated with AD, or regulation of gene expression or protein function of a gene or protein associated with AD are tested in studies to demonstrate the safety and/or efficacy of the agents (e.g., regulatory trials such as Food and Drug Administration trials). In some embodiments, the agents are sold for the purpose of treating or preventing AD or related conditions. In some such embodiments, the agents are labeled with instruction for use or with other labels (e.g., labeling required by a government agency such as the Food and Drug Administration). In some embodiments the agents are marketed (e.g., advertised) for use in treating or preventing AD or related conditions.

The present invention also provides methods for identifying compounds involved in the modulation of a symptom of AD, regulation of a biological pathway associated with AD, or regulation of gene expression or protein function of a gene or protein associated with AD, whereby an agent is first tested for a biological activity prior to treating a cell or organism with the agent to identify modulation of a symptom of AD, regulation of a biological pathway associated with AD, or regulation of gene expression or protein function of a gene or protein associated with AD.

The present invention also contemplates the design of APP processing modulating compounds that readily traverse the blood brain barrier. Brain uptake of drugs can be improved via prodrug formation. Prodrugs are pharmacologically inactive compounds that result from transient chemical modifications of biologically active species. The chemical change is usually designed to improve some deficient physicochemical property, such as membrane permeability or water solubility. After administration, the prodrug, by virtue of its improved characteristics, is brought closer to the receptor site and is maintained there for longer periods of time. Here it gets converted to the active form, usually via a single activating step. For example, esterification or amidation of hydroxy-, amino-, or carboxylic acid- containing drugs, may greatly enhance lipid solubility and, hence, entry into the brain. Drugs may be adapted for CNS delivery through the use of lipophilic analogs, liposomes, PEGylated derivitives, immunoliposomes, redox delivery systems, carrier mediated delivery systems, receptor or vector mediated delivery, osmotic blood brain barrier disruption, biochemical blood brain barrier disruption, or olfactory delivery. Alternatively, delivery could also be achieve via invasive procedures such as intraventricular or intrathecal delivery, injections, catheters, pumps, biodegradable polymer wafers, microspheres, nanoparticles, or delivery from biological tissues. (Mishra, A. et al., 2003, J Pharm Pharmaceut Sci, 6(2):252-273). Additionally, compounds or intervention may be applied with the compounds of the present invention to increase update to desired tissues. Such methods include, but are not limited to, those described in U.S. patent application. Ser. Nos. 20030162695, 20030129186, 20020038086, and 20020025313, herein incorporated by reference in their entireties.

### DESCRIPTION OF THE FIGURES

FIG. 1 shows the mevolonate pathway which involves the biosynthesis of various lipid compounds and protein prenylation.

### GENERAL DESCRIPTION OF THE INVENTION

In experiments conducted during the development of the present invention, the approaches of using Drosophila melanogaster (hereafter referred to as Drosophila) as a system in which to study human disease-associated networks and of genetic screens were combined to identify modifiers of AD phenotypes.

One of the most profound and surprising biological discoveries in the last two decades is that most animals across the animal kingdom, including humans, possess many of the same genes that function in similar ways in cells, tissues and organs. In fact, only 94 of an estimated 1,278 human gene families are vertebrate-specific. Furthermore, at least 77% of known human disease genes have at least one counterpart within the genome of Drosophila, a model organism and workhorse in the study of genetics (Reiter et al., Gen. Res. 111:1114; Table 1 (2001)). Many genes implicated in human diseases, including signaling pathways and effectors of tissue- and cell-specification, were originally identified and characterized in the fruit fly. Thus, genes within most human disease-associated networks are present in the fruit fly genome and have comparable roles in fly biology.

TABLE 1
Drosophila shares many important aspects of
biology and disease pathways with humans

| | |
|---|---|
| Genes shared between | |
| humans & Drosophila | Human disease relevance |

Signaling pathway

| | |
|---|---|
| Notch, presenilin, APP | Alzheimer's disease; leukemia |
| Hedgehog, ptc | Basal cell carcinoma; |
| | medulloblastoma |
| Insulins, InR, PI3K, PDK | Diabetes |
| TGF-beta, Wnt | Colon cancer |
| G-protein coupled receptors | Obesity/diabetes; hypertension |

Tissue formation

| | |
|---|---|
| SREBP, PPAR-gamma | Obesity/diabetes |
| MyoD, Mef | Muscular dystrophy; cardiomyopathy |
| Pax-6 | Aniridia |

Cell structural/biological components

| | |
|---|---|
| p53, Akt, Rb, Abl, EGF-R | Transformation & malignancy |
| KCNQ1, KCNH2, SCN5A | Long-QT syndrome |
| KCNQ3, BFNC2, EBN1, KCNQ2 | Neonatal epilepsy |
| PKD1 | Polycystic kidney disease |
| Alpha-syn, parkin, UCHL-1 | Parkinson's disease |

These striking parallels in biological processes among animals are reflected in commercial applications for methods of treatment for human conditions. For example, the protein products of the Transforming Growth Factor-beta (TGF-beta) gene family act as signaling molecules and regulate diverse biological activities (Hogan, Curr. Op. Gen. Dev. 6:432 (1996)). One subset of this family, the Bone Morphogenetic Proteins (BMPs), is characterized by its ability to induce bone formation, both when added to or expressed in cultured cells and when implanted in animals (Sampath et al., Proc. Natl. Acad. Sci. USA 90:6004 (1993)). This has been exploited in a medical device (FDA ref. no. H010002) in which the BMP protein OP-1 is indicated for use as an alternative to autograft in recalcitrant long bone nonunions. The Drosophila counterpart of the OP-1 protein, called 60A, exhibits a very similar biological activity in rats and is sufficient to induce bone formation within dose ranges that have been reported for OP-1 (Sampath et al., 1993). It is therefore expected that Drosophila nucleic acids, their encoded proteins, and the networks within which they interact will have biological activities almost identical to their human homologs. Hence, it is advantageous to use the strengths of Drosophila as an experimental system to study human disease gene-associated networks in genetic modifier screens.

Genetic screens are used to discover genes that carry out various biological activities (St. Johnston, Nat. Rev. Gen. 3:176 (2002)). A change in the activity of a gene in an organism, either through its loss of function or over-expression, can cause a detectable phenotype by disrupting normal biological processes. As described in detail below, the present invention provides a number of genes whose expression is correlated to the regulation of AD phenotypes.

The screens used during the development of the present invention, as described in the examples below, provide unparalleled capability for identifying genes and proteins with desired in vivo activities. In particular, the present invention employs a collection of genetically modified organisms that statistically represent at least one organism that overexpresses each gene of the genome. These animals can be crossed against a disease model (e.g., an animal expressing a human gene associated with disease) to determine which of the genes of the genome alter the phenotype of the model. Thus, a comprehensive survey of the in vivo activity of each gene in the genome is provided. Unlike existing high-throughput in vitro methods (microarrays, proteomic methods, twin-hybrid systems), the methods used in the present invention provide high-throughput in vivo results. While in vitro approaches have successfully generated dense arrays of data, they lack a critical ability to discriminate among the hundreds or thousands of identified targets because they do not assess their functional relevance.

Genetic modifier screens, such as those of the present invention, offer a superior alternative to other systems because they identify only genes of biological relevance. Genetic modifier screens are used to test interactions among genes that act together within networks to carry out various biological activities. A change in the activity of a gene in an organism, either through its loss of function or mis-expression, can cause a detectable phenotype by disrupting normal biological processes. A change in the activity of another gene that acts together within a gene network with the first will often detectably modify this phenotype by either enhancing or suppressing it. Changes in the activity of genes that do not genetically interact with the first gene will not specifically modify the phenotype. Genetic modifier screens hold a crucial advantage over yeast two-hybrid or proteomics systems in their ability to detect interactions among genes whose products may not physically interact. Furthermore, they are far better than DNA microarray technologies because genetic modifier screens identify interactions of biological importance, not just associations between gene expression patterns and different cell- or tissue-types.

The present invention arose from large-scale genetic modifier screening to identify Drosophila strains with mutations that modify the processing of APP. By determining the genomic location of the EP insertion in these strains the mutated gene causing the modified phenotype was identified. The Drosophila experimentation provided genetic evidence of pathways with heretofore-unknown involvement in APP processing. Once the identity of the Drosophila gene was determined, computer searching of publicly available genome databases readily confirmed the presence and identity of corresponding human genes. Studies were then performed to confirm cleavage of APP in model mammalian cells and that the corresponding enzyme activity could be reduced with compounds known to interfere with the newly discovered molecular target. Thus, the present invention sets forth novel molecular targets and agents that can interfere with APP processing. The newly discovered disease targets enable those skilled in the art to utilize compounds (e.g., antibodies, antisense molecules, small molecule inhibitors, etc.) directed at these targets and discover new chemical compositions through drug screening, design chemical libraries targeted to specific enzyme active sites, design chemical compositions through rational design methods, design nucleic acid molecules to reduce gene expression, and produce other medicaments. Such compounds may be formulated into medicaments for pharmacological uses in humans or other animals. The present invention also contemplates the use of such agents as medicaments for the treatment of AD and other diseases that arise from APP processing. In preferred embodiments, for use in the treatment of diseases of the central nervous system (such as AD), the compounds of the present invention are adapted to pass from blood to the cerebral spinal fluid and brain or are formulated for direct administration to tissues of the central nervous system.

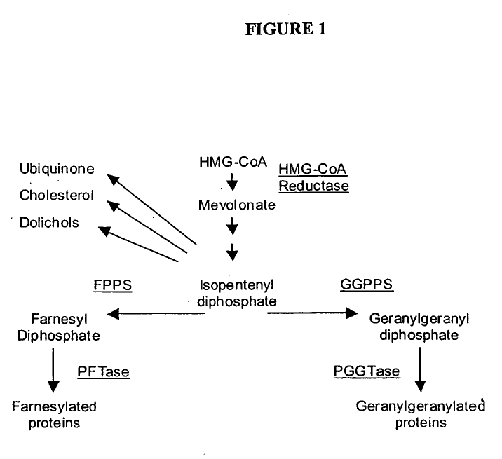

In the present invention we have found a novel association of the mevolonate pathway in APP processing. The mevolonate pathway involves the biosynthesis of various lipid compounds and protein prenylation (shown in FIG. 1). Protein prenylation has been found to be critical for the function of key proteins involved in signal transduction (Casey, P. J. and Seabra, M. C. J. Biol. Chem. 1996, 271, 5289-5296; Marshall, C. J. Science 1993, 259, 1865-1866). Prenylation is a form of lipid modification in which either a C-15 famesyl or C-20 geranylgeranyl group is covalently attached via a thioether linkage to the cysteine residue of proteins near the carboxy terminus. A variety of proteins are modified at their carboxy terminus by isoprenylation, including heterotrimeric G protein gamma subunits (Yamane, H. K. et al. Proc. Natl. Acad. Sci. USA 1991; 88, 286-290; Fukada, Y. et al. J. Biol. Chem. 1994, 269, 5163-5170), low molecular weight GTPases (Takai, Y., et al. Int. Rev. Cytol. 1992, 133, 187-230), protein kinases (Takai, Y., et al. Int. Rev. Cytol. 1992, 133, 187-230), nuclear lamins (Farnsworth, C. C. et al., J. Biol. Chem. 1989, 264, 20422-20429), and viral proteins (Glenn, J. S. et al., Science 1992, 256, 1331-1333). Enzymes such as HMG-CoA reductase, farnesyl diphosphatesynthetase (FPPS), and genanylgeranyldiphosphatesynthetase (GGPPS) are known to act within the mevolonate pathway. Three different isoprenyltransferases have been identified which modify a variety of eukaryotic proteins; protein farnesyltransferase (PFTase), protein geranylgeranyltransferase type I (PGGTase-I), and type II-geranylgeranyltransferase. It is also known that molecules such as ubiquinone, cholesterol and dolichols arise from the mevolonate pathway (See FIG. 1).

The epsilon-4 allele of the apolipoprotein E gene (APOE), which is involved in the CNS distribution of cholesterol among neurons, represents the most common lipoprotein expressed in the brain and is the main genetic risk factor for sporadic AD. APOE-4 is associated with a slight increase of serum cholesterol and might have a direct role in the deposition of amyloid fibrils and Abeta aggregation. Clinical studies and animal experimentation have indicated a beneficial effect of cholesterol-lowering drugs in AD, yet the relevant molecular mechanism remains unknown. Another class of compounds, bisphosphonates, is known to interfere with FPPS and is useful medically in the treatment of osteoporosis (additionally these drugs are being tested clinically for the treatment of various cancers). Other compounds have been shown to interfere with the action of PFTase and PGGTase, resulting in reduced protein prenylation. Although targeting cholesterol biosynthesis with statin drugs has been shown to modulate APP cleavage, the results have not indicated that enzymes involved in protein prenylation are involved (Park, In-Ho, et al. 2003, Neurobiology of Aging, 24:637-643). None of the compounds that interfere with protein prenylation pathway enzymes have heretofore been shown to modulate APP processing nor have they been considered as possible treatments for AD.

Definitions

To facilitate understanding of the invention, a number of terms are defined below.

The phrase “modulation of a symptom of AD” refers to detectable changes in a symptom associated with AD brought about by an intervention (e.g., exposure to an agent). Symptoms may be observed, for example, in patients, in animal models, and in cells or tissue. Symptoms include, but are not limited to, dementia, memory loss, language deterioration, impaired visuospatial skills, poor judgment, indifferent attitude, etc. Symptoms also include biological events associated with AD, including, but not limited to, formation or existence of proteinaceous filaments (e.g., comprising extracellular amyloid senile plaques and intraneuronal neurofibrillary tangles).

The phrase “regulation of a biological pathway associated with AD” refers to an activation or repression of any biological pathway associated with AD. Biological pathways associated with AD include, but are not limited to, molecular and cellular processes involving presenilins, ApoE, APP protein, and gamma secretase.

The phrase “regulation of gene expression or protein function of a gene or protein associated with AD” refers to activation or repression of gene expression (e.g., directly or indirectly) or protein activity (directly or indirectly) of a gene or protein associated with AD. Genes and proteins associated with AD include, but are not limited to, presenilins, ApoE, APP protein, and gamma secretase.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

Where amino acid sequence is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “polymerase chain reaction” (“PCR”) refers to the method of K.B. Mullis U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “antisense” is used in reference to RNA sequences that are complementary to a specific RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced into an embryo, this transcribed strand combines with natural mRNA produced by the embryo to form duplexes. These duplexes then block either the further transcription of the MRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand. Regions of a nucleic acid sequences that are accessible to antisense molecules can be determined using available computer analysis methods.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific MRNA sequence encoding a specific protein, are found in the cell as a mixture with numerous other mRNAs that encode a multitude of proteins. However, isolated nucleic acid encoding a gene includes, by way of example, such nucleic acid in cells ordinarily expressing the gene where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “coding region” when used in reference to structural gene refers to the nucleotide sequences that encode the amino acids found in the nascent polypeptide as a result of translation of a MRNA molecule. The coding region is bounded, in eukaryotes, on the 5′ side by the nucleotide triplet “ATG” that encodes the initiator methionine and on the 3′ side by one of the three triplets which specify stop codons (i.e., TAA, TAG, TGA).

As used herein, the term “purified” or “to purify” refers to the removal of contaminants from a sample. For example, antibodies are purified by removal of contaminating non-immunoglobulin proteins; they are also purified by the removal of immunoglobulin that does not bind a target of interest. The removal of non-immunoglobulin proteins and/or the removal of immunoglobulins that do not bind the target results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “recombinant DNA molecule” as used herein refers to a DNA molecule that is comprised of segments of DNA joined together by means of molecular biological techniques.

The term “transgene” as used herein refers to a foreign gene that is placed into an organism by introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term host cell refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The terms “overexpression” and “overexpressing” and grammatical equivalents, are used in reference to levels of MRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic MRNA.

The term “test compound” or “agent” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

### DETAILED DESCRIPTION OF THE INVENTION

The present invention relates to newly identified molecular pathways involved in the processing of APP and the use of molecules along such pathways that are targets for therapeutic intervention. The invention also relates the correlation between the expression of genes and AD. The invention also relates to modifying the activity of a protein that affects AD by regulating the expression of the nucleic acids, homologs, or active variants or their encoded proteins. The present invention also encompasses methods for screening for agents that inhibit or potentiate action of a target gene or protein. The present invention also relates to methods for screening for susceptibility to AD or AD-related conditions.

The present invention provides methods and compositions for using the genes and gene products as targets for screening drugs, other agents, or stimuli that alter AD or biological pathways or phenotypes associated with AD or otherwise modulates a desired phenotype (e.g., disease phenotype).

The present invention further provides pharmaceutical compositions that modulate the processing of APP which may comprise all or portions of polynucleotide sequences, polypeptides, inhibitors or antagonists of bioactivity, including antibodies, alone or in combination with at least one other agent, such as a stabilizing compound, and may be administered in any sterile, biocompatible pharmaceutical carrier, including, but not limited to, saline, buffered saline, dextrose, and water.

The methods of the present invention find use in treating diseases or altering physiological states. Peptides can be administered to the patient intravenously in a pharmaceutically acceptable carrier such as physiological saline. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, and intraperitoneal. Therapeutic administration of a polypeptide intracellularly can also be accomplished using gene therapy as described above.

As is well known in the medical arts, dosages for any one patient depends upon many factors, including the patient's size, body surface area, age, the particular compound to be administered, sex, time and route of administration, general health, and interaction with other drugs being concurrently administered.

Accordingly, in some embodiments of the present invention, nucleotide and amino acid sequences can be administered to a patient alone, or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert. In another embodiment of the present invention, polynucleotide sequences or amino acid sequences may be administered alone to individuals subject to or suffering from a disease or condition (e.g., AD).

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks′ solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. For example, an effective amount of the pharmaceutical agent may be that amount that regulates AD symptoms or phenotypes associated with AD. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).

Pharmaceutical preparations which can be used orally include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition. For polynucleotide or amino acid sequences, conditions indicated on the label may include treatment of condition related to apoptosis.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration range.

A therapeutically effective dose refers to that amount of which ameliorates symptoms of the disease state or condition. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds which exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference). Those skilled in the art will employ different formulations for the polypeptide or nucleic acid (e.g., of Table 2) than for the inhibitors of polypeptide or nucleic acid expression.

Experimental

The following examples are provided in order to demonstrate and further illustrate certain preferred embodiments and aspects of the present invention and are not to be construed as limiting the scope thereof.

### EXAMPLE 1

### Identification of Genes that Modify Gamma-Secretase Activity

The ability to ectopically express nucleic acids in a conditional, tissue-specific manner is an important tool in Drosophila biology. One method, used widely by those skilled in the art, is the Gal4-UAS system (Brand and Perrimon, Development 118:401 (1993)). Briefly, this system comprises genetically crossing two different transgenic Drosophila lines that carry transposable elements, called P-elements (or “EP”) (U.S. Pat. No. 4,670,388), within their genomes. A P-element insertion in one line contains a nucleic acid fragment encoding the yeast Gal4 transcriptional activator protein. Gal4 protein can be expressed either by an endogenous promoter in the Drosophila genome upstream of the insertion site of a P-element carrying the Gal4 open reading frame and a minimal promoter, or by a regulatory element that is engineered into the P-element upstream of the Gal4 open reading frame and a minimal promoter. In the former case Gal4 protein is expressed in the pattern of the endogenous enhancer, while in the latter, Gal4 protein is expressed in the pattern of the regulatory element placed upstream of it. The second transgenic Drosophila line carries a P-element containing a tandem array of fourteen upstream activating sequence (UAS) sites upstream of a nucleic acid encoding a gene of interest. One embodiment of this latter type of P-element that is commonly used by those skilled in the art has the UAS sites upstream of an hsp70 promoter and a multiple cloning site to facilitate the insertion of a gene of interest and is referred to as pUAST (Brand and Perrimon, 1993). A genetic cross of the two Drosophila lines results in the inheritance of both kinds of P-elements in a fraction of their progeny. In these progeny, the Gal4 protein is locally mis-expressed in some tissues, in which it binds to the UAS sites in the second P-element, resulting in expression of the gene of interest. This system exhibits a tremendous amount of flexibility in both the patterns of Gal4 expression lines available, and in being able to engineer pUAST constructs containing any gene of interest.

One of methods used to perform genetic modifier screens known to those skilled in the art employs a library of EP fly lines (Rorth et al., Development 125:1049 (1998)). EP lines each contain a genomic insertion of a P-element, called EP element, which contains fourteen tandem copies of the upstream activator sequences (UAS) from yeast immediately upstream of a basal promoter. This sequence is bound with high affinity by the Gal4 transcriptional activator protein. The insertion of an EP element thus places a Gal4-inducible promoter either (1) near the 5′ (upstream) end of a gene or (2) within a gene. Wherever Gal4 protein is present within the fly, genes into which an EP element has inserted will either be (1) activated or (2) inactivated via expression of an anti-sense RNA depending on the orientation of an EP element insertion into or near a gene. The insertion can in some cases cause a targeted gene's loss of function by disrupting gene structure, even in the absence of Gal4.

We adopted the EP library screen method to identify genes regulating gamma-secretase activity. One of the primary biological activities of the gamma-secretase complex is the cleavage of the basement of the transmembrane domain of APP. To screen for modifiers of gamma-scretase activity, we used a Drosophila line that mis-expresses a chimeric form of Drosophila homolog of human APP protein, hereafter referred to as APPL-SV. APPL-SV comprises a nucleic acid encoding the Drosophila homolog of human APP (APP-like, or APPL; Rosen et al., Proc. Natl. Acad. Sci. USA 86:2478 (1989)) fused to the DNA binding domain of the Suppressor of hairless transcription factor (Su(H)), followed by a domain of the VP16 protein that is often used to confer very strong transcriptional activity to heterologous transcription factors (the latter two protein domains are together referred to hereafter as SV).

APPL-SV was generated by:

1) digesting a pGEX-Su(H) plasmid (Bailey and Posakony, Genes Dev. 9:2609 (1995)) with the restriction endonucleases NcoI and PstI (both available from MBI Fermentas), separating the DNA fragments by size using gel electrophoresis (Sambrook and Russell, Molecular Cloning: A Laboratory Manual, the third edition, Cold Spring Harbor Laboratory Press, NY (2001)), and isolating an approximately 1,050 base pair fragment containing the Su(H) DNA binding domain.

2) The VP16 activation domain was PCR amplified from the plasmid pAct-GAL4-VP16 (Han and Manley, Genes Dev. 7:491 (1993)) with the following primers: 5′-AAA CTG CAG GCG CCC CCC CGA CCG ATG TCA GC-3′ SEQ ID NO:1 and 5′-GCT CTA GAG TTT ATT GTG GAT ACG AA-3′ SEQ ID NO:2, and this approximately 300 base pair PCR fragment was size-separated via gel electrophoresis and then digested with PstI and XbaI (MBI Fermentas).

3) Using T4 DNA ligase (MBI Fermentas), the NcoI/PstI-cut Su(H) DNA binding domain and PstI/XbaI-cut VP16 activation domain were then ligated into Litmus 28 plasmid (New England BioLabs) digested with NcoI and XbaI (Sambrook and Russell, 2001) to form Litmus 28-SV.

4) Litmus 28 was digested with EcoRI endonuclease (MBI Fermentas) and NcoI.

5) The expression tag sequence clone GH04413 (found in the GenBank database hosted by the National Center for Biotechnology Information) containing the nucleic acid sequence encoding APPL was digested with EagI (New England BioLabs) and EcoRI endonucleases, the DNA size-separated via gel electrophoresis, and an approximately 2,700 base pair fragment containing APPL isolated.

6) The GH04413 clone was also PCR amplified using the following primers: 5′-GTT CGC GCA ACA TGC ACA-3′ SEQ ID NO:3 and 5′-ATG CCA TGG CTC CGC CCT CTT TCA CTT CGA AAT AC-3′ SEQ ID NO:4, and digested with EagI and NcoI.

7) The EcoRI/NcoI-cut Litmus 28 plasmid from step 4, the EagI/EcoRI APPL restriction fragment from step 5, and the EagI/NcoI-cut APPL PCR fragment from step 6 were ligated together to form Litmus 28-APPL.

8) Litmus 28-SV was digested with NcoI and XbaI, and the DNA fragment containing SV was isolated via gel electrophoresis.

9) Litmus 28-APPL was digested with EcoRI and NcoI, and the DNA fragment containing APPL was isolated via gel electrophoresis.

10) The nucleic acid fragments from steps 8 and 9 were ligated together into pUAST plasmid (Brand and Perrimon, 1993) digested with EcoRI and XbaI to generate UAS-APPL-SV.

Transgenic flies containing UAS-APPL-SV were generated by injection of recombinant DNA at a concentration of 1 μg/μL into Drosophila melanogaster embryos of the genotype w¹¹¹⁸ according to standard procedures well-known to those skilled in the art (Spradling, Drosophila: A Practical Approach, D. B. Roberts, ed., IRL Press, DC (1986), pp. 175-197).

The expression of APPL-SV was driven in the Drosophila wing via the Gal4-UAS system using an engrailed Gal4 driver that expresses Gal4 in the posterior compartments of developing segments and appendages in Drosophila and is known to those skilled in the art as e16E-Gal4 (Ye and Fortini, 1999). APPL-SV can be used to measure PS activity as follows. When expressed in Drosophila cells, the APPL-SV protein is thought to undergo cleavage of extracellular portion, producing a shorter protein (Luo et al., J. Neurosci. 10:3849 (1990); Torroja et al., J. Neuro. 16:4638 (1999)) fused to SV, which is tattered in plasma cell membrane. Gamma-secretase protein activity can cleave and release this shorter chimeric protein from the membrane. This cleavage product can then translocate to the nucleus where the Su(H)-VP16 transcriptional activation domain chimera will activate targets of Su(H), mimicking the effects of Notch signaling (Furriols and Bray, Dev. Biol. 227:520 (2000)), and thus inducing a gain of function Notch-like vein phenotypes (Matsuno et al., Development 121:2633 (1995)). Vein phenotypes are thereby used to monitor the activity of gamma-secretase activity in cleaving APPL-SV. Adult wing vein phenotypes are very commonly used among those skilled in the art to measure the biological activity of genes, including Notch signaling (e.g., Matsuno et al., 1995), because they are easily detected under a microscope and are dispensable for fly viability. Individual wing veins can be easily distinguished from each other and have names that are familiar to those skilled in the art.

In general, the following nomenclature for fly lines is used hereafter: “name of Gal4 driver”:“name of gene expressed under Gal4 control.” If the expression of two or more genes is being driven by Gal4 via UAS sites, then the genes will be separated also by a colon (i.e., Gal4 driver:gene 1:gene 2:gene 3: etc.). Two homologous chromosomes are distinguished by ‘/’. None homologous chromosomes are separated by a semicolon.

e16E:APPL-SV causes a moderate truncation of the L5 vein in the in the adult Drosophila wing around the posterior cross vein (“PCV”). In order to sensitize the effect of APPL-SV cleavage, we inactivated one copy of Presenilin gene (Psn) by introducing a Psn null mutation (Psn^{B3}), resulting in the fly genotype of e16E:APPL-SV/CyO; Psn^{B3}/MKRS. Psn is the core catalytic component of gamma-secretase complex. The files with this genotype have little longer L5 vein than e16E:APPL-SV/CyO; +/+ flies because of the reduced gamma-secretase activity. The end of L5 veins of e16E:APPL-SV/CyO; Psn^{B3}/MKRS flies are in the range from 1/3 to 1/2 of the length between PCV and the wing margin. The CyO and MKRS chromosomes are easily recognizable under stereomicroscopes by curled up wings and short truncated bristles, respectively.

Because the flies with the e16E:APPL-SV reporter gene have shorter L5 vein when they have higher gamma-secretase activity, overexpression or antisense expression of modifiers of gamma-secretase should also modify this phenotype. Therefore, such modifiers were screened for by crossing e16E:APPL-SV/CyO; Psn^{B3}/MKRS flies to each of approximately 26,500 EP lines, which are already mapped for the locations of EP element insertion site by GenExel, Inc. (http://genexel.com/eng/htm/genisys.htm). The L5 vein lengths of the progenies resulting from these crosses, which have one copy of e16E:APPL-SV reporter, Psn^{B3}mutation, and the gene trapped by an EP element, were compared to that of e16E:APPL-SV/+; Psn^{B3}/+ flies. e16E:APPL-SV;EP flies that exhibited either much more severe (i.e., enhanced) truncations of the L5 vein or suppression of the original e16E:APPL-SV phenotype (i.e. restoration of the phenotype towards a normal-length L5 vein) were considered to reflect insertions of EP elements into genes that interact with gamma-secretase. e16E:APPL-SV;EP flies that exhibited only additive vein effects compared with the e16E:APPL-SV and e16E:EP phenotypes alone were considered to reflect EP insertions into genes that do not interact with gamma-secretase. 112 EP lines met the criteria as the enhancers of the e16E:APPL-SV/+; Psn^{B3}/+ vein phenotype and 223 EP lines met the criteria as the suppressors of the e16E:APPL-SV/+; Psn^{B3}/+ vein phenotype.

We focused on target genes that up-regulate gamma-secretase activity that fulfilled the following criteria (accordingly 14 EP lines were selected from 335 primary candidates identified in the genetic screen):
1. The gene trapped by the EP element encodes a metabolic enzyme or protein modification enzyme.
2. When the gene is over-expressed by the EP element driven by the e16E-Gal4 driver, the phenotype should be enhanced.
3. When the gene is knocked-down by anti-sense expression driven by a e16E-Gal4 driver, the phenotype should be suppressed.
   We also excluded genes that are elsewhere well characterized and believed not to be regulators of gamma-secretase.

Gamma-secretase cleaves Notch as well as APP. This is a big disadvantage of gamma-secretase for a therapeutic target of Alzheimer's disease. Therefore, we generated a chimeric Notch reporter gene, named N™-SV, in order to identify the EP lines which specifically enhance APP cleavage but not Notch cleavage. N™-SV is identical with APPL-SV except the APPL sequence was replaced with truncated Notch sequence which contains only the transmembrane domain and the intracellular domain of Notch. Cleavage of this reporter in the wing where e16E-gal4 is expressed results in the same L5 vein truncation phenotype as APPL-SV.

Generation of N™-SV. NTM-SV was generated by: 1) Digesting a pMtNMG (N-minigene as described in Wharton, K. A., et al., Cell, 43:567-581, 1985) with the restriction endonucleases AatII and SacII (both available from MBI Fermentas), separating the DNA fragments by size using gel electrophoresis (Sambrook and Russell, Molecular Cloning: A Laboratory Manual, the third edition, Cold Spring Harbor Laboratory Press, NY, 2001), and isolating an approximately 3,076 base pair fragment containing the intracellular domain. 2) The transmembrane domain of Notch was PCR amplified from the plasmid pMtNMG with the following primers: 5′-CGG GAT CCC ACG GCG GCC AAA CAT CAG CT-3′, SEQ ID NO:5, and 5′-TTG GCC GTG TGG ATC ACG TC-3′, SEQ ID NO:6 and this approximately 280 base pair PCR fragment was size-separated via gel electrophoresis and then digested with BamHI and AatII (MBI Fermentas). 3) Using T4 DNA ligase (MBI Fermentas), the AatII/SacII-cut Notch intracellular domain and BamHI/AatII-cut transmembrane domain were then ligated into Litmus 28-N^{m}SG plasmid (as described in Ju, B. G., et al., Nature, 405:191, 2000) digested with BamHI and SacII (Sambrook and Russell, 2001) to form Litmus 28-N^{ΔECN}. 4) Litmus 28-SV (for construct methods see Step 3 above for the generation of APPL-SV) was digested with BglII endonuclease (MBI Fermentas) and NcoI. 5) Litmus 28-N^{ΔECN} was digested with BglII (MBI Fermentas) and SalI endonucleases, the DNA size-separated via gel electrophoresis, and an approximately 510 base pair fragment containing both signal peptide and transmembrane of Notch. 6) The plasmid pMtNMG was also PCR amplified using the following primers: 5′-GGC ACA TGG CGT CAC CTG-3′, SEQ ID NO:7 and 5′-ATT TGC GGC CGC CAT GGC GCC ACC ATC CTG ATG CGC-3′, SEQ ID NO:8 and digested with SalI and NcoI. 7) The BglII/NcoI-cut Litmus 28-SV from step 4, the BglII/SalI N™ restriction fragment from step 5, and the SalI/NcoI-cut N PCR fragment from step 6 were ligated together to form Litmus 28-N™-SV. 8) Litmus 28-N™SV was digested with BglII and XbaI, and the DNA fragment containing N™-SV was isolated via gel electrophoresis. 9) The nucleic acid fragments from steps 8 were ligated into pUAST plasmid (Brand and Perrimon, 1993) digested with BglII and XbaI to generate UAS-N™-SV.

Transgenic flies containing UAS-N™SV were generated by injection of recombinant DNA at a concentration of 1 μg/μL into Drosophila melanogaster embryos of the genotype w1118 according to standard procedures well-known to those skilled in the art (Spradling, Drosophila: A Practical Approach, D. B. Roberts, ed., IRL Press, DC (1986), pp. 175-197).

We crossed the 14 EP lines selected from the primary genetic screen with e16E:N™-SV/CyO; Psn^{B3}/MKRS flies in parallel with e16E:APPL-SV/CyO; Psn^{B3}/MKRS flies. From this test, only two EP lines did not enhance the truncated L5 vein phenotype of e16E:N™-SV/+; Psn^{B3}/+ flies while they still enhanced the phenotype of e16E:APPL-SV/+; Psn^{B3}/+ flies. One of these two EP lines is GE13720, which has an EP element in the 5′ untranslated region of the first exon of farnesyl pyrophosphate synthase (FPPS). The orientation of the EP element in this EP line is to cause overexpression of FPPS when it combined with a Gal4 driver.

To confirm the modifier effect of FPPS on gamma-secretase activity, we choose EP line GE13823 that contains an EP element in the second exon of FPPS gene in the direction opposite from that of FPPS gene transcription. Thus, it is expected that EP driven transcription would produces antisense RNA of FPPS when the flies were crossed with a Gal4 driver. As expected, GE13823 crossed with e16E:APPL-SV/CyO; Psn^{B3}/MKRS resulted in suppressed L5 vein phenotype of e16E:APPL-SV/+; Psn^{B3}/+ in the progenies. This confirms that knock-down of FPPS by antisense RNA reduces the gamma-secretase activity. The phenotypes of w¹¹¹⁸ control flies and GE13823 having one copy of e16E:APPL-SV reporter gene with one copy of Psn^{B3} mutation are summarized in Table 2 and Table 3.

TABLE 2
Determination of wing vein phenotype in w¹¹¹⁸ control flies

| | | Maximum | |
|---|---|---|---|
| Fly Number | L5 Length¹ | L5 Length² | Percent³ |
| 1 | 86 | 471 | 18 |
| 2 | 0 | 507 | 0 |
| 3 | 0 | 507 | 0 |
| 4 | 0 | 514 | 0 |
| 5 | 0 | 514 | 0 |
| 6 | 0 | 486 | 0 |
| 7 | 0 | 500 | 0 |
| 8 | −21 | 500 | −4 |
| 9 | 0 | 500 | 0 |
| 10 | 71 | 464 | 15 |
| 11 | 164 | 450 | 37 |
| 12 | 121 | 429 | 28 |
| 13 | 186 | 429 | 43 |
| 14 | 36 | 500 | 7 |
| 15 | 71 | 464 | 15 |
| 16 | 50 | 471 | 11 |
| 17 | 0 | 493 | 0 |
| 18 | 114 | 429 | 27 |
| 19 | 43 | 500 | 9 |
| 20 | 100 | 479 | 21 |
| 21 | 21 | 464 | 5 |
| 22 | 0 | 486 | 0 |
| | | Average | 10.5 |
| | | Standard deviation | 13.4 |

¹The values shown are the lengths in micrometers of the L5 vein from the position of the PCV as they extend toward the wing margin. Negative values indicate the distance of the tip of the L5 vein proximal to the PVC.
²The values shown are the distances in micrometers between the PCV and the wing margin (along the normal track of the L5 vein) and represent the maximum possible length of the L5 vein.
³The values shown are “L5 Length” divided by “Maximum L5 Length” as percent.

TABLE 3
Determination of wing vein phenotype
in GE13823 flies (FPPS antisense)

| | | Maximum | |
|---|---|---|---|
| Fly Number | L5 Length¹ | L5 Length² | Percent³ |
| 1 | 229 | 443 | 52 |
| 2 | 236 | 407 | 58 |
| 3 | 243 | 436 | 56 |
| 4 | 271 | 464 | 58 |
| 5 | 221 | 421 | 53 |
| 6 | 257 | 407 | 63 |
| 7 | 57 | 407 | 14 |
| 8 | 0 | 414 | 0 |
| 9 | 271 | 436 | 62 |
| 10 | 279 | 421 | 66 |
| 11 | 164 | 407 | 40 |
| 12 | 229 | 400 | 57 |
| 13 | 264 | 400 | 66 |
| 14 | 279 | 393 | 71 |

| | |
|---|---|
| Average | 51.1 |
| Standard deviation | 20.3 |

¹The values shown are the lengths in micrometers of the L5 vein from the position of the PCV as they extend toward the wing margin.
²The values shown are the distances in micrometers between the PCV and the wing margin (along the normal track of the L5 vein) and represents the maximum possible length of the L5 vein.
³The values shown are “L5 Length” divided by “Maximum L5 Length” as percent.

### Measuring APP Cleavage in Mammalian Cells

The studies described above clearly show that overexpression of FPPS in GE13720 results in enhanced enzymatic cleavage APP. The presence of APP cleavage products has been shown to correspond with AD. The role of FPPS activity can be further established by measuring APP cleavage in mammalian cells. For such measurement a cell line was constructed: 1) that expressed the Swedish mutant form of human APP and human BACE, and; 2) wherein APP cleavage through the action of beta and gamma secretase could be quantified. Human BACE cDNA was cloned from HEK293 cell using a forward primer (5′-GGC GAA TTC GTG CCG ATG TAA CGG GCT CCG -3′, SEQ ID NO:9) and a backward primer (5′-GGC CTC GAG CTG GAA CCC ACC TTG CCA GCC-3′, SEQ ID NO:10) corresponding to the human BACE sequence. Each primer contained EcoRI and XhoI enzyme digestion sites at the ends of each primer to facilitate vector insertion. After cleavage of human BACE cDNA and pcDNA3.1/Myc-His(+)A (Invitrogen, Carlsbad, Calif.) with EcoRI and XhoI enzymes, two fragments were ligated, allowing for the expression of human BACE in pcDNA3.1/Myc-His(+)A vector.

To make an expression construct of APP, we used plasmid CB6-swAPP695 containing the cDNA sequence of the Swedish mutant form of APP695 (as described in Neuroscience Letters, 1997, 235:1-4). pCB6-swAPP695 was first digested with XbaI, then partially digested with BamHI, and fragments isolated containing the full-length swAPP695 sequences with terminal BamHI and XbaI sites (partial digestion with Bam HI was necessary because of an internal BamHI site). The swAPP695 fragments were ligated into pcDNA3.1/hygro mammalian expression vector (Invitrogen, Carlsbad, Calif.) DNA after its digestion with BamHI and XbaI.

BACE cDNA in pcDNA3.1/Myc-His(+)A was transfected into HEK293 cell line with 5 microgram/ml lipofectamine (Invitrogen). 400 microgram/ml of G418 was used for neomycin selection since pcDNA3.1/Myc-His(+)A vector contains neomycin resistant gene. Human swAPP695 cDNA in pcDNA3.1/hygro was also transfected into BACE overexpressing HEK293 cells with 5 microgram g/ml lipofectamine (Invitrogen). As a selection marker for the vector, 200 microgram g/ml of hygromycin was used.

### Inhibition of APP Cleavage with Prenylation Inhibitors

The modified HEK293 cell line described herein provides a highly relevant, sensitive assay system to determine if compounds that modulate FPPS and corresponding prenylation enzymes affect APP cleavage. Accordingly, this assay system was used to evaluate four compounds with known activities against FPPS for their ability to inhibit APP cleavage (compounds are shown in Table 4).

TABLE 4
Test compounds that inhibit FPPS activity¹

| Compound | | | Molecular | Working |
|---|---|---|---|---|
| Code | Compound Name | Compound Formula | Weight | Concentration^{3,4} |
| A | alendronate sodium¹ | C4H12NNaO7P2.3H2O | 325 | 1.8 |
| B | etidronatic acid² | C2H8O7P2 | 206 | 2.0 |
| C | risedronate sodium¹ | C7H10NO7P2Na.5/2H2O | 350 | 0.3 |
| D | zolendronic acid¹ | C5H10N2O7P2.H20 | 290 | 0.1 |

¹Compounds were purchased from Pharm Chemical, Shanghai Lansheng Corporation, Shanghai, China and a certificate of analysis was provided indicating compound identify and purity in excess of 97%.
²Compound was purchased from Sigma Chemical Co., St. Louis, MO; Cat. No. H6773, a 60% aqueous solution.
³millimolar
⁴pH of working solution was adjusted to 7.0

Test compounds were serially diluted in water and added to approximately 300,000 NEK293 cells in 60 mm dishes overexpressing BACE/swAPP. After 3 days fresh culture medium was added along with the test compounds. After a total of 6 days, the medium from each dish was collected to measure Abeta 1-40 level using an enzyme immunoassay kit (Biosource, Camarillo Calif., Cat. No. KHB3481). The data in Table 5 show that FPPS inhibiting compounds reduce the amount of APP cleaved in a dose dependent manner.

TABLE 5
Reduction of APP cleavage with FPPS inhibiting compounds¹

| | |
|---|---|
| Compound | Dilution of Test Compound |

| Code | 10⁻³ | 10⁻⁴ |
|---|---|---|
| A | 47.9 | 58.4 |
| B | 34.4 | 49.6 |
| C | 59.4 | 64.0 |
| D | ns² | 1.9 |
| ¹Numerical values shown are picograms per milliliter of Abetal-40 as measured by enzyme immunoassay at 6 days of culture with test compounds. Although the action of beta and gamma secretase results in the production of both Abeta40 and Abeta42, Abeta 40 is the | | |
| # predominant form and thus is more readily quantified by enzyme immunoassay. Test compounds were diluted to by the factors shown from the working concentrations listed in Table 4. Control wells with no test compound yielded 107.1 picograms per milliliter of Abetal-40 measured by enzyme immunoassay. | | |

²ns; results are not shown due to poor viability of HEK293 cells in culture at the indicated compound concentration. It is expected that test compounds are toxic at high concentrations and corresponding APP cleavage measurements would be prone to error.

We claim:

1. A method for modulating APP processing in a mammalian cell, comprising administering an FPPS antagonist to said cell.

2. The method of claim 1, wherein the FPPS antagonist is an acid or salt form of alendronate sodium, etidronatic acid, risedronate sodium, or zolendronic acid.

3. The method of claim 1, wherein the antagonist is in the form of a medicament suitable for mammalian administration.

4. The method of claim 3, wherein the medicament is adapted to enhance passage of the medicament from blood to cerebrospinal fluid.

5. The method of claim 1, wherein the medicament is a nucleic acid able to reduce cellular expression of the FPPS protein.

6. The method of claim 5, wherein the nucleic acid is an antisense molecule or a small interfering RNA molecule.

7. The method of claim 1, wherein the antagonist is a low molecular weight compound, peptide, protein, lipid, phospholipid, carbohydrate, glycolipid, or glycoprotein.

8. A method for reducing APP cleavage in a mammalian cell comprising the step of exposing a mammalian cell to a protein prenylation antagonist.

9. The method of claim 8, wherein a molecular target of the medicament of is FPPS, geranylgeranyl protein transferase and/or geranylgeranyldipiphosphate synthase.

10. A method for treating mammalian subjects providing:
a. a subject with symptoms or risk factors consistent with a disease associated with abnormal APP processing
b. an antagonist of FPPS
c. administering said inhibitor of FPPS to said subject as to reduce APP processing in said subject.

11. A method for treating or preventing Alzheimer's disease in humans providing:
a. a human subject with symptoms or risk factors consistent with Alzheimer's disease
b. an antagonist of FPPS
c. administering said antagonist of FPPS to said human subject as to reduce APP processing in said subject.

12. A method for treating mammalian subjects providing:
a. a subject with symptoms or risk factors consistent with a disease associated with abnormal APP processing
b. an antagonist of protein prenylation
c. administering said antagonist of protein prenylation to said subject as to reduce APP processing in said subject.

13. A method for treating or preventing Alzheimer's disease in humans providing:
a. a human subject with symptoms or risk factors consistent with Alzheimer's disease
b. an antagonist of protein prenylation
c. administering said antagonist of protein prenylation to said human subject as to reduce APP processing in said subject.


SEQUENCE LISTING (10 sequences)

SEQ ID NO: 1 - DNA, 32 bp, Artificial Sequence
  Synthetic
aaactgcaggcgcccccccgaccgatgtcagc

SEQ ID NO: 2 - DNA, 26 bp, Artificial Sequence
  Synthetic
gctctagagtttattgtggatacgaa

SEQ ID NO: 3 - DNA, 18 bp, Artificial Sequence
  Synthetic
gttcgcgcaacatgcaca

SEQ ID NO: 4 - DNA, 35 bp, Artificial Sequence
  Synthetic
atgccatggctccgccctctttcacttcgaaatac

SEQ ID NO: 5 - DNA, 29 bp, Artificial Sequence
  Synthetic
cgggatcccacggcggccaaacatcagct

SEQ ID NO: 6 - DNA, 20 bp, Artificial Sequence
  Synthetic
ttggccgtgtggatcacgtc

SEQ ID NO: 7 - DNA, 18 bp, Artificial Sequence
  Synthetic
ggcacatggcgtcacctg

SEQ ID NO: 8 - DNA, 36 bp, Artificial Sequence
  Synthetic
atttgcggccgccatggcgccaccatcctgatgcgc

SEQ ID NO: 9 - DNA, 30 bp, Artificial Sequence
  Synthetic
ggcgaattcgtgccgatgtaacgggctccg

SEQ ID NO: 10 - DNA, 30 bp, Artificial Sequence
  Synthetic
ggcctcgagctggaacccaccttgccagcc